(19) United States
(12) Patent Application Publication
(10) Pub. No.: US 2022/0115145 A1
(43) Pub. Date: Apr. 14, 2022

(54) METHOD, DEVICE, AND EQUIPMENT FOR USER GROUPING, AND COMPUTER-READABLE STORAGE MEDIUM

(71) Applicant: Ping An Technology (Shenzhen) Co., Ltd., Shenzhen (CN)

(72) Inventors: Tiange CHEN, Shenzhen (CN); Yuan ZHANG, Shenzhen (CN)

(73) Assignee: Ping An Technology (Shenzhen) Co., Ltd., Shenzhen (CN)

(21) Appl. No.: 17/533,471

(22) Filed: Nov. 23, 2021

Related U.S. Application Data
(63) Continuation of application No. PCT/CN2020/124389, filed on Oct. 28, 2020.

(30) Foreign Application Priority Data
Sep. 8, 2020 (CN) 202010937805.4

(51) Int. Cl. G16H 50/70; G06F 16/28; G16H 10/20; G06Q 10/06

(52) U.S. Cl. CPC G16H 50/70; G06Q 10/06313; G16H 10/20; G06F 16/285

(57) ABSTRACT

A method, device, equipment for user grouping, and a non-transitory computer-readable storage medium are provided, which are applicable to the field of medical technology. The method includes the following. Net benefits of multiple users in a target project are obtained. According to the net benefits of the multiple users in the target project and a solution of the target project, a net-benefit coefficient corresponding to the solution is determined. For each grouping variable of the target project, a fluctuation value corresponding to the grouping variable is determined according to the net-benefit coefficient. According to a grouping variable with the largest fluctuation value, the multiple users are divided into multiple user groups. For each user group obtained by division, users in the user group are divided according to a fluctuation value corresponding to each grouping variable of the target project, until a user group meeting a preset condition is obtained.

### CROSS-REFERENCE TO RELATED APPLICATION(S)

This application is a continuation under 35 U.S.C. § 120 of International Application No. PCT/CN2020/124389, filed on Oct. 28, 2020, which claims priority under 35 U.S.C. § 119(a) and/or PCT Article 8 to Chinese Patent Application No. 202010937805.4, filed on Sep. 8, 2020, the entire disclosures of which are hereby incorporated by reference.

### TECHNICAL FIELD

This disclosure relates to the technical field of artificial intelligence, and particularly to a method, device, and equipment for user grouping, and a computer-readable storage medium.

### BACKGROUND

At present, users need to be grouped in some scenarios, so that user group analysis, message pushing aiming at different user groups, and the like can be implemented based on user grouping to achieve precise marketing and so on. However, the inventor found in research that the existing user grouping methods have limitations and one-sidedness. For example, in personalized medicine, users are generally grouped based on only treatment effectiveness, which leads to a relatively low reliability of grouping. Therefore, how to achieve reliable user grouping has become a technical problem to-be-solved.

### SUMMARY

In a first aspect of the disclosure, a method for user grouping is provided. Net benefits of a plurality of users in a target project are obtained. According to the net benefits of the plurality of users in the target project and a solution of the target project, a net-benefit coefficient corresponding to the solution is determined. For each grouping variable of the target project, a fluctuation value corresponding to the grouping variable is determined according to the net-benefit coefficient. The plurality of users are divided into a plurality of user groups according to a grouping variable with the largest fluctuation value. For each user group obtained by division, a fluctuation value corresponding to each grouping variable of the target project is determined according to a net-benefit coefficient and users in the user group are divided according to a grouping variable with the largest fluctuation value, until a user group meeting a preset condition is obtained.

In a second aspect of the disclosure, an equipment for user grouping is provided. The equipment includes a processor and a memory. The memory is coupled with the processor, and configured to store computer programs. The computer programs include program instructions which are called by the processor and cause the processor to carry out the following actions. Net benefits of a plurality of users in a target project are obtained. According to the net benefits of the plurality of users in the target project and a solution of the target project, a net-benefit coefficient corresponding to the solution is determined. For each grouping variable of the target project, a fluctuation value corresponding to the grouping variable is determined according to the net-benefit coefficient. The plurality of users are divided into a plurality of user groups according to a grouping variable with the largest fluctuation value. For each user group obtained by division, a fluctuation value corresponding to each grouping variable of the target project is determined according to a net-benefit coefficient and users in the user group are divided according to a grouping variable with the largest fluctuation value, until a user group meeting a preset condition is obtained.

In a third aspect of the disclosure, a non-transitory computer-readable storage medium is provided. The non-transitory computer-readable storage medium stores computer programs. The computer programs include program instructions which, when executed by a processor, cause the processor to carry out all or part of the operations of the method in the first aspect of the disclosure.

### BRIEF DESCRIPTION OF THE DRAWINGS

In order to describe technical solutions of implementations of the disclosure more clearly, the following will give a brief description of accompanying drawings used for describing the implementations. Apparently, accompanying drawings described below are merely some implementations. Those of ordinary skill in the art can also obtain other accompanying drawings based on the accompanying drawings described below without creative efforts.

FIG. 1 is a schematic flowchart illustrating a method for user grouping provided in implementations of the disclosure.

FIG. 2 is a schematic flowchart illustrating a method for user grouping provided in other implementations of the disclosure.

FIG. 3 is a schematic structural diagram illustrating a device for user grouping provided in implementations of the disclosure.

FIG. 4 is a schematic structural diagram illustrating an equipment for user grouping provided in implementations of the disclosure.

FIG. 5 is a schematic structural diagram illustrating a system for user grouping provided in implementations of the disclosure.

### DETAILED DESCRIPTION

Hereinafter, technical solutions of implementations of the disclosure will be described in a clear and comprehensive manner with reference to accompanying drawings intended for the implementations. It is evident that the implementations described herein constitute merely some rather than all the implementations of the disclosure. Those of ordinary skill in the art will be able to derive other implementations based on these implementations without making creative efforts, which all such derived implementations shall all fall within the protection scope of the disclosure.

The technical solutions of the disclosure may be applicable to the technical field of artificial intelligence, digital healthcare, smart city, block-chain, and/or big data, to achieve accurate user grouping. Optionally, data involved, such as a net benefit and/or a grouping variable, may be stored in a database or a block-chain, which is not limited in the disclosure.

The technical solutions of the disclosure may be applicable to a device for user grouping to achieve user grouping. Optionally, the device for user grouping may be a terminal, a server, or a data platform or other equipment. The terminal herein may include a mobile phone, a tablet computer, a computer, etc., which is not limited in the disclosure. It can be understood that in other implementations, the terminal may also have other names, for example, the terminal is also called a terminal equipment, a smart terminal, a user equipment, a user terminal, etc., which is not exhaustively listed herein.

The technical solutions of the disclosure may be applicable to the technical field of artificial intelligence, smart city, block-chain and/or big data. The technical solutions of the disclosure may be achieved through a data platform or other equipment. The data involved may be stored through a block-chain node, or stored in a database, which is not limited in the disclosure.

At present, users need to be grouped in many scenarios. User grouping refers to dividing users into groups according to a specific condition (or attribute). After grouping, a variety of analysis and operations aiming at different user groups can be performed, for example, pushing messages to users in a same user group, analyzing characteristics of users in a user group with the best condition, providing a same solution for users in a same user group, or the like. However, the inventor found that the existing user grouping schemes have a problem of low reliability. For example, personalized medicine refers to implementing the best diagnosis and treatment for an individual patient according to evidence-based medicine in the context of big data, so that the patient can achieve a relatively optimal prognosis level. The personalized medicine can be achieved by identifying which type of individual population is suitable for this treatment plan by means of real-world clinical data. However, the existing algorithms generally take only treatment effectiveness as a goal, without considering an economic burden and side effects caused by the treatment, etc., which leads to limitations and one-sidedness in treatment plan recommendation. As a result, the reliability of user grouping is reduced. In the disclosure, a grouping variable for grouping is determined based on fluctuation corresponding to net benefits and a solution of a project, to achieve user grouping. As such, accurate grouping can be achieved based on the net benefits obtained and the solution, thereby improving the reliability of user grouping.

According to implementations of the disclosure, a method, device, equipment, and system for user grouping, and a medium are provided, which can improve reliability of user grouping. The implementations of the disclosure will be described in detail below.

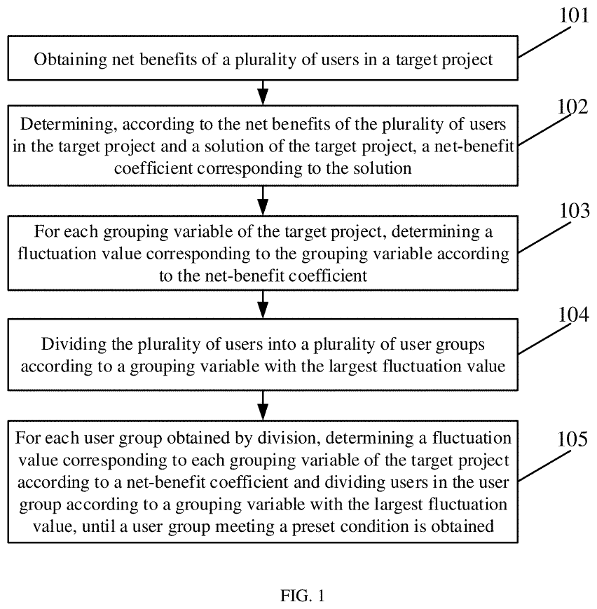

FIG. 1 is a schematic flowchart illustrating a method for user grouping provided in implementations of the disclosure. The method is performed by the above device for user grouping (e.g., a server). As illustrated in FIG. 1, the method includes the following.

At 101, net benefits of multiple users in a target project are obtained.

The net benefit herein, also known as net income or other names, represents a benefit obtained by subtracting a cost from income. For details of the net benefit, reference can be made to related descriptions of cost-effectiveness. The net benefit obtained may be a net benefit in a target disease.

Optionally, the net benefits can be obtained in a variety of ways. For example, the net benefits are calculated in real time based on a predetermined algorithm, or obtained from a storage device such as a block-chain node, etc., which is not limited in the disclosure.

In some implementations, the net benefits are calculated in real time based on a formula corresponding to the type of the target project. As an example, a net benefit corresponding to user i can be determined based on NBᵢ=W*QALYᵢ−Cᵢ, where NB represents the net benefit; QALY represents a quality-adjusted life year, which is a measure that combines the quantity and the quality of life lived, and measures the quantity of life lived after factors (e.g., health damage, chronic conditions, disability, etc.) affecting the quality of life lived are adjusted; W represents the price that the user is willing to pay for health (measured by QALY); C represents an economic cost of the solution.

In some implementations, the net benefits are obtained from a block-chain. That is, a net benefit of each user in the target project can be stored in the block-chain in advance. The net benefits of the users are obtained from the block-chain, which can improve reliability of the net benefits obtained, and accordingly, reliability of user grouping based on the net benefits obtained can be improved. As an example, the device for user grouping sends a net-benefit obtaining request carrying an identification of the target project to a block-chain node. The block-chain node searches net benefits corresponding to the identification of the target project after the net-benefit obtaining request is received and identity verification of the device for user grouping passes, and then returns the net benefits to the device for user grouping. The device for user grouping receives the net benefits sent by the block-chain node.

In some implementations, the net benefits are obtained from a server. As an example, a net-benefit obtaining request carrying an identification of the target project is sent to a server to request net benefits corresponding to the target project. The manner of requesting net benefits from a server is similar to the manner of requesting net benefits from a block-chain node, which will not be repeated herein.

In some implementations, the device for user grouping stores net benefits of different users in each project, so that the net benefits of the users in the target project can be searched based on an identification of the target project. Optionally, the device for user grouping is a node of the block-chain or a node outside the block-chain.

At 102, according to the net benefits of the multiple users in the target project and a solution of the target project, a net-benefit coefficient corresponding to the solution is determined.

In some implementations, the device for user grouping determines, according to the net benefits of the multiple users in the target project and the solution of the target project, the net-benefit coefficient corresponding to the solution as follows. A net-benefit parameter model is fitted in all groups. The net-benefit coefficient corresponding to the solution is obtained by processing the net benefits in the target project and the solution of the target project with the net-benefit parameter model. The all groups are user groups before the first division. Optionally, the net-benefit parameter model is a regression model.

In some implementations, the device for user grouping determines, according to the net benefits of the multiple users in the target project and the solution of the target project, the net-benefit coefficient corresponding to the solution as follows. A net-benefit coefficient table is obtained, where the net-benefit coefficient table represents a correspondence among net benefits in a project, a solution of the project, and a net-benefit coefficient. According to the net benefits in the target project and the solution of the target project, the net-benefit coefficient corresponding to the solution is determined from the net-benefit coefficient table. Optionally, the net-benefit coefficient table may be stored locally, stored in a block-chain (e.g., the net-benefit coefficient table is obtained from a block-chain node), stored on a server outside the block-chain, or the like, which is not limited in the disclosure.

It can be understood that a project may correspond to one or more solutions. As an example, the project of the disclosure is embodied as a disease (or the type of a disease), and the solution is embodied as a treatment plan for the disease. As another example, the project is embodied as a problem, and the solution is embodied as an answer to the problem. The project and the solution are not limited in the disclosure.

At 103, for each grouping variable of the target project, a fluctuation value corresponding to the grouping variable is determined according to the net-benefit coefficient.

The grouping variable herein refers to a variable that affects an effect of the solution. For example, the target project is embodied as a disease and the solution is embodied as a treatment plan for the disease, the grouping variable is a variable that affects a treatment effect for the disease. The fluctuation value herein is indicative of a degree of stability of a grouping variable, or indicative of a degree of instability of the grouping variable relative to a target variable (the net benefits). Optionally, the larger the fluctuation value (or an absolute value of the fluctuation value) is, the more unstable the grouping variable is relative to the net benefits.

In some implementations, the device for user grouping determines, according to a pre-stored correspondence between projects and grouping variables, multiple grouping variables corresponding to the target project. Optionally, the correspondence may be stored in a form of a table (or list), an array, a matrix, or the like, which is not limited in the disclosure. Optionally, the correspondence may be stored locally, stored in a block-chain, stored in a server, or the like, which is not limited in the disclosure.

In some implementations, the device for user grouping determines the fluctuation value corresponding to each grouping variable as follows. For each grouping variable of the target project, the fluctuation value corresponding to the grouping variable is determined according to the net-benefit coefficient, the net benefits, and a fluctuation function. The fluctuation value is indicative of a degree of instability of the grouping variable relative to the net benefits. The fluctuation function can be defined based on a net benefit parameter and a net-benefit coefficient parameter.

In some implementations, the device for user grouping determines the fluctuation value corresponding to each grouping variable as follows. For each grouping variable of the target project, the fluctuation value corresponding to the grouping variable is determined from a stored fluctuation-value table based on the net-benefit coefficient obtained, the net benefits obtained, the grouping variable, and the solution. The fluctuation-value table may include a net-benefit coefficient in a project, net benefits in the project, a solution of the project, and fluctuation values corresponding to grouping variables of the project.

At 104, the multiple users are divided into multiple user groups according to a grouping variable with the largest fluctuation value.

After the fluctuation value corresponding to each grouping variable of the target project is obtained, the grouping variable with the largest fluctuation value can be determined. That is, the grouping variable with the largest degree of instability (the most unstable grouping variable relative to the net benefits) can be determined. Further, the multiple users are divided according to the grouping variable with the largest degree of instability to obtain the multiple user groups. The multiple users may be divided into two user groups (i.e., subgroups), three user groups, or the like.

In some implementations, the device for user grouping determines, according to a greedy algorithm, a grouping critical value corresponding to the grouping variable with the largest fluctuation value. The device for user grouping divides the multiple users according to the grouping critical value to obtain the multiple user groups (e.g., two user groups).

In some implementations, the device for user grouping divides the multiple users according to an intermediate value of the grouping variable to obtain the multiple user groups (e.g., two user groups), or divides the multiple users according to two endpoint values of the grouping variable to obtain the multiple user groups (e.g., two or three user groups, and the obtained user groups are equal or approximately equal in terms of the number of users).

At 105, for each user group obtained by division, a fluctuation value corresponding to each grouping variable of the target project is determined according to a net-benefit coefficient and users in the user group are divided according to a grouping variable with the largest fluctuation value, until a user group meeting a preset condition is obtained.

After the multiple users are divided according to the grouping variable with the largest degree of instability to obtain the multiple user groups, for each user group obtained, the operations at 103-105 are repeatedly performed to iterate until a preset stopping standard is reached.

Optionally, the preset condition includes at least one of the following. A significance value corresponding to a fluctuation value is greater than a significance threshold. The number of user groups obtained is greater than a first number threshold. The number of users in at least one of the user groups obtained is less than a second number threshold. Based on the above, when the preset condition is satisfied during iteration of user grouping, the iteration is stopped, and multiple user groups divided are obtained.

It can be understood that users in the same user group having the same net benefit referred to herein means that differences between net benefits of the users in the same user group do not exceed a threshold. As an example, for each of the multiple user groups divided, net benefits of users in the user group are all the same or within a same range, that is, these net benefits are basically the same.

Optionally, after user grouping is completed, operations such as message pushing, user characteristic analysis, and so on may be performed based on the user groups divided, which is not limited in the disclosure.

In some implementations, the target project is a target disease, and the solution of the target project is a treatment plan for the target disease. After the user group meeting the preset condition is obtained, the device for user grouping further determines a user group with the highest net benefit from all user groups obtained, and recommends the treatment plan to users in the user group with the highest net benefit.

In the implementations of the disclosure, according to the net benefits of the multiple users in the target project and the solution of the target project, the device for user grouping determines the net-benefit coefficient corresponding to the solution. For each grouping variable of the target project, the device for user grouping determines a fluctuation value corresponding to the grouping variable according to the net-benefit coefficient. The device for user grouping divides the multiple users according to fluctuation values determined. For each user group obtained by division, users in the user group are divided according to a fluctuation value corresponding to each grouping variable of the target project, until the user group meeting the preset condition is obtained, so as to achieve user grouping. As such, accurate user grouping can be achieved based on the solution and the net benefits obtained, thereby improving the reliability of user grouping.

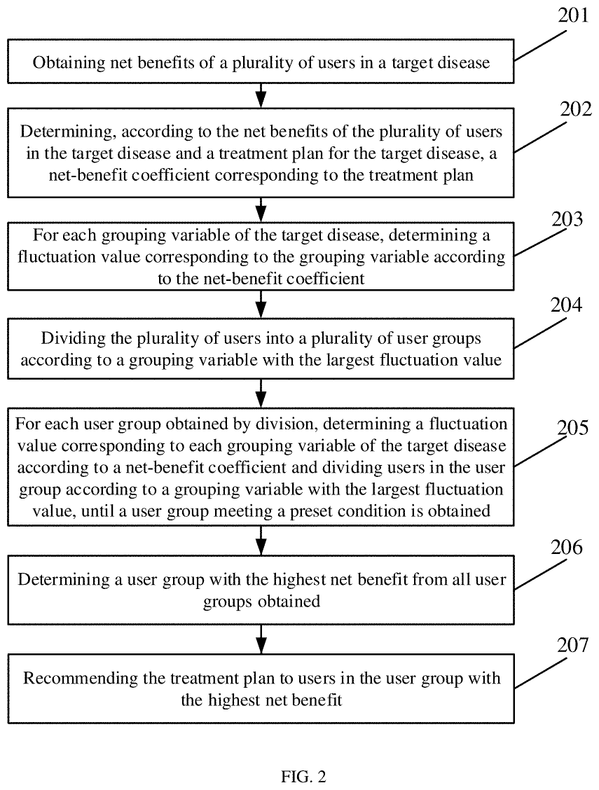

FIG. 2 is a schematic flowchart illustrating a method for user grouping provided in other implementations of the disclosure. The project is embodied as a disease and the solution of the project is embodied as a treatment plan for the disease, as illustrated in FIG. 2, the method includes the following.

At 201, net benefits of multiple users in a target disease are obtained.

Optionally, the net benefits can be determined in a variety of ways. For example, the disease is breast cancer, and taking breast cancer screening as an example, a net benefit of user i can be defined as follows:

NBᵢ=W*QALYᵢ−Cᵢ

where NB represents the net benefit; QALY represents a quality-adjusted life year, which is a measure that combines the quantity and the quality of life lived, and measures the quantity of life lived after factors (e.g., health damage, chronic conditions, disability, etc.) affecting the quality of life lived are adjusted; W represents the price that the user is willing to pay for health (measured by QALY), and W generally has a value of $50000/QALY in the world, that is, willing to pay $50000 for an additional 1 quality-adjusted life year; C represents an economic cost of the treatment plan.

For a breast cancer patient, his quality of life is generally considered to be 0.7 of the quality of life in a healthy state, because his life is affected by pain, side effects caused by treatment, etc. The following will give a simplified example. If breast cancer patient 1 treated with targeted therapy spends $200,000 and survives for 10 years, then his QALY=10*0.7, C=$200000, NB=10*0.7*50000−200000=150000. If untreated breast cancer patient 2 survives for 5 years only, then his QALY=5*0.7, C=0, NB=5*0.7*50000=175000.

At 202, a net-benefit coefficient corresponding to the treatment plan is determined according to the net benefits of the multiple users in the target disease and the treatment plan for the target disease.

In some implementations, the net-benefit coefficient θ is determined by fitting a net-benefit parameter model in all users (e.g., n users).

As an example, net benefit NBᵢ of user i (i=1, . . . , n) is used as a dependent variable and treatment plan Tᵢ is used as an independent variable, a regression model is established as follows:

NBᵢ=f(Tᵢ*θ)

where parameter θ represents a net-benefit coefficient obtained in treatment plan T, and a formula for solving the parameter is as follows:

\hat{θ}=argmin Σᵢ₌₁ⁿΨ(NBᵢ,θ)

Based on the above, the net-benefit coefficient \hat{θ} can be determined.

In some implementations, the net-benefit coefficient is obtained by looking up a table. As an example, a correspondence between disease types and net-benefit coefficients is stored in advance. A net-benefit coefficient can be determined quickly according to the net benefits, a disease type corresponding to the treatment plan, and the correspondence.

At 203, for each grouping variable of the target disease, a fluctuation value corresponding to the grouping variable is determined according to the net-benefit coefficient.

The grouping variable herein refers to a variable that affects a treatment effect for the disease. The fluctuation value herein is indicative of a degree of stability of a grouping variable, or indicative of a degree of instability of the grouping variable relative to a target variable (the net benefits).

Optionally, the grouping variables can be determined based on the target disease. As an example, a correspondence between an identification of a disease and a grouping variable (a disease may correspond to multiple grouping variables) can be set in advance. A grouping variable(s) corresponding to the target disease is determined according to the correspondence. Optionally, the grouping variables can be determined based on a disease type of the target disease. As an example, a corresponding between disease types and grouping variables can be set in advance (a disease type may correspond to multiple grouping variables). A disease type to which the target disease belongs is determined. A grouping variable(s) corresponding to the target disease is determined based on the disease type and the correspondence.

For example, for breast cancer patients, grouping variables may include age, childbearing history, pathological stage, tumor volume, metastatic characteristic (i.e., metastasize or not), gene phenotype, etc., to achieve precise treatment recommendation.

In some implementations, after the net-benefit coefficient \hat{θ} is determined, the parameter \hat{θ} estimated is substituted into a fluctuation function to verify instability of the parameter \hat{θ} relative to each grouping variable Zⱼ (j=1, . . . , J, where J represents the number of grouping variables involved). For example, the fluctuation function is as follows:

$${W}_{j} ( t , \hat{θ} ) = {\sum }_{i = 1}^{⌊ nt ⌋} \frac{\partial Ψ ( {NB}_{{Z}_{ij}} , \hat{θ} )}{\partial \hat{θ}} ( 0 \leq t \leq 1 )$$

if the fluctuation function Wⱼ randomly fluctuates around 0, it indicates that the parameter estimation is relatively stable with respect to the grouping variable Zⱼ; if the fluctuation function has a systematic deviation from 0, it indicates that instability is high. As an example, an absolute value of Wⱼ(t,\hat{θ}) is used as the fluctuation value. The larger a fluctuation value, the lower stability of a grouping variable corresponding to the fluctuation value, so as to determine a grouping variable with the largest degree of instability, that is, the grouping variable corresponding to the largest absolute value of Wⱼ(t,\hat{θ}).

In other implementations, a degree of instability of each grouping variable relative to the target variable (the net benefits), such as the fluctuation value, may also be determined based on other methods, which is not limited in the disclosure.

At 204, the multiple users are divided into multiple user groups according to a grouping variable with the largest fluctuation value.

At 205, for each user group obtained by division, a fluctuation value corresponding to each grouping variable of the target disease is determined according to a net-benefit coefficient and users in the user group are divided according to a grouping variable with the largest fluctuation value, until a user group meeting a preset condition is obtained. That is, the operations at 203-205 are repeatedly performed to iterate until a preset stopping standard is reached.

Optionally, when dividing the multiple users, the multiple users are divided according to a greedy algorithm, that is, all possible values are tried to find the best grouping critical value, to divide the multiple users into two subgroups. Optionally, the multiple users are divided in other ways. As an example, after determining the group variable with the largest degree of instability (e.g., the group variable with the largest fluctuation value), the multiple users are divided into two subgroups through the greedy algorithm.

For example, after calculation, a degree of instability of the parameter \hat{θ} relative to the tumor volume is the highest, then according to a critical value (10 cm²) of the tumor volume, the multiple users are divided into a user group with tumor volume <10 cm² and a user group with tumor volume [symbol] 10 cm² (i.e., subgroups). Further, for each subgroup obtained, a grouping variable (e.g., “metastasize or not”) with the largest fluctuation value is determined, and users in the subgroup are further divided into a subgroup with “metastasized” and a subgroup with “not metastasized” based on the grouping variable “metastasize or not”. The operations of dividing users in the subgroup according to a grouping variable with the largest fluctuation value are performed repeatedly until the preset stopping standard is reached.

Optionally, the stopping standard may be that a significance value P of the fluctuation function is greater than 0.95, the number of subgroups obtained is greater than a first number threshold, or the number of users in at least one of the subgroups obtained is less than a second number threshold, or the like, which is not limited in the disclosure.

The multiple users can finally be divided into multiple subgroups based on the foregoing method, and net benefits of users in the same subgroup against treatment plan T are basically the same. Moreover, some subgroups have a relatively high net benefit against treatment plan T, and some subgroups have a relatively low net benefit against treatment plan T.

At 206, a user group with the highest net benefit is determined from all user groups obtained.

At 207, the treatment plan for the target disease is recommended to users in the user group with the highest net benefit.

As an example, treatment plan T for a subgroup with a high net benefit is recommend to patients in the subgroup. As such, it is possible to decide whether to recommend treatment plan T to an individual patient or recommend other treatment plans to the patient based on an accurate grouping result. Thus, accurate user grouping can be achieved, thereby improving reliability of treatment plan recommendation.

In some implementations, after grouping is completed, grouping information and a corresponding treatment plan can be bound and uploaded to a block-chain. As an example, information of patients in a subgroup with a high net benefit and a corresponding treatment plan are bound and uploaded to the block-chain. In this way, when recommending a treatment plan to a patient in future, a treatment plan corresponding to the patient (i.e., the treatment plan corresponding to a high net benefit) can be obtained from the block-chain, which can improve safety of information recommendation while improving reliability of treatment plan recommendation.

In the implementation of the disclosure, according to the net benefits of the multiple users in the target disease and the treatment plan for the target disease, the device for user grouping determines the net-benefit coefficient corresponding to the treatment plan. For each grouping variable of the target disease, the device for user grouping determines a fluctuation value corresponding to the grouping variable according to the net-benefit coefficient. The device for user grouping divides the multiple users according to fluctuation values determined. For each user group obtained by division, users in the user group are divided according to a fluctuation value corresponding to each grouping variable of the target disease, until the user group meeting the preset condition is obtained, so as to achieve user grouping. In the implementations of the disclosure, accurate grouping is implemented according to whether the multiple users can obtain benefits through the treatment plan, where users having the closest net benefits are classified into a group. As such, a group of users capable of obtaining the highest benefit through the treatment plan can be found. The technical solutions of the disclosure may be applicable to a hospital clinical decision support system, to provide a doctor with a recommendation for the most cost-effective treatment in line with health economics, and to select the most cost-effective treatment for a patient under the premise of providing an effective treatment, which can reduce burden of the patient and medical insurance.

It can be understood that the foregoing method implementations are illustrative examples of the method for user grouping of the disclosure, and the description of each implementation has its own emphasis. For the parts not described in detail in one implementation, reference may be made to related descriptions in other implementations.

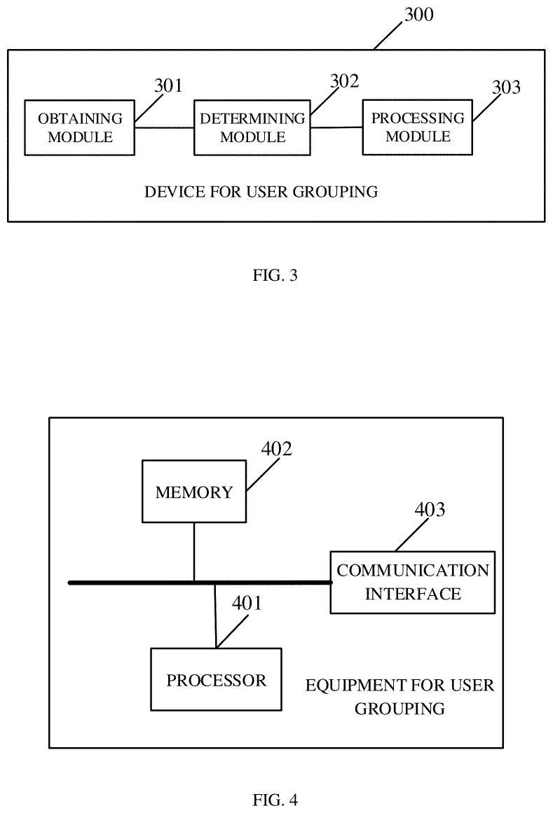

According to implementations of the disclosure, a device for user grouping is further provided. The device for user grouping includes a module configured to perform the method described with reference to FIG. 1 or FIG. 2. FIG. 3 is a schematic structural diagram illustrating a device for user grouping provided in implementations of the disclosure. The device for user grouping of these implementations may be configured in a server. As illustrated in FIG. 3, a device 300 for user grouping includes an obtaining module 301, a determining module 302, and a processing module 303. The obtaining module 301 is configured to obtain net benefits of multiple users in a target project. The determining module 302 is configured to determine, according to the net benefits of the multiple users in the target project and a solution of the target project, a net-benefit coefficient corresponding to the solution. The determining module 302 is further configured to determine, according to the net-benefit coefficient, a fluctuation value corresponding to each grouping variable of the target project. The processing module 303 is configured to divide the multiple users into multiple user groups according to a grouping variable with the largest fluctuation value. For each user group obtained by division, the determining module 302 is configured to determine a fluctuation value corresponding to each grouping variable of the target project according to a net-benefit coefficient and the processing module 303 is configured to divide users in the user group according to a grouping variable with the largest fluctuation value, until a user group meeting a preset condition is obtained.

In some implementations, the determining module 302 configured to determine, according to the net benefits of the multiple users in the target project and the solution of the target project, the net-benefit coefficient corresponding to the solution is configured to: fit a net-benefit parameter model in all groups, where the all groups are user groups before the first division, and the net-benefit parameter model is a regression model; and obtain the net-benefit coefficient corresponding to the solution by processing the net benefits in the target project and the solution of the target project with the net-benefit parameter model.

In some implementations, the determining module 302 configured to determine, according to the net benefits of the multiple users in the target project and the solution of the target project, the net-benefit coefficient corresponding to the solution is configured to: obtain a net-benefit coefficient table from a block-chain, where the net-benefit coefficient table represents a correspondence among net benefits in a project, a solution of the project, and a net-benefit coefficient; and determine the net-benefit coefficient corresponding to the solution from the net-benefit coefficient table, according to the net benefits in the target project and the solution of the target project.

In some implementations, the determining module 302 is further configured to determine, according to a pre-stored correspondence between projects and grouping variables, multiple grouping variables corresponding to the target project. The determining module 302 configured to determine, for each grouping variable of the target project, the fluctuation value corresponding to the grouping variable according to the net-benefit coefficient is configured to: determine the fluctuation value corresponding to each of the multiple grouping variables of the target project, according to the net-benefit coefficient, the net benefits, and a fluctuation function, where the fluctuation value is indicative of a degree of instability of the grouping variable relative to the net benefits.

In some implementations, the processing module 303 configured to divide the multiple users into the multiple user groups according to the grouping variable with the largest fluctuation value is configured to: determine a grouping critical value corresponding to the grouping variable with the largest fluctuation value according to a greedy algorithm; and divide the multiple users into the multiple user groups according to the grouping critical value.

In some implementations, the preset condition includes at least one of the following. A significance value corresponding to a fluctuation value is greater than a significance threshold. The number of user groups obtained is greater than a first number threshold. The number of users in at least one of the user groups obtained is less than a second number threshold.

In some implementations, the target project is a target disease, and the solution of the target project is a treatment plan for the target disease. The determining module 302 is further configured to determine a user group with the highest net benefit from all obtained user groups after obtaining, by the processing module 303, the user group meeting the preset condition, where users in the same user group have the same net benefit. The processing module 303 is further configured to recommend the treatment plan to users in the user group with the highest net benefit.

It can be understood that functional modules of the device for user grouping in these implementations can be implemented according to the method of the foregoing method implementations described with reference to FIG. 1 or FIG. 2. For a specific implementation process, reference may be made to related descriptions of the method of the foregoing method implementations described with reference to FIG. 1 or FIG. 2, which will not be repeated herein.

In implementations of the disclosure, according to the net benefits of the multiple users in the target project and the solution of the target project, the device for user grouping determines the net-benefit coefficient corresponding to the solution. For each grouping variable of the target project, the device for user grouping determines a fluctuation value corresponding to the grouping variable according to the net-benefit coefficient. The device for user grouping divides the multiple users according to fluctuation values determined. For each user group obtained by division, users in the user group are divided according to a fluctuation value corresponding to each grouping variable of the target project, until the user group meeting the preset condition is obtained, so as to achieve user grouping. As such, accurate user grouping can be achieved based on the solution and the net benefits obtained, thereby improving the reliability of user grouping.

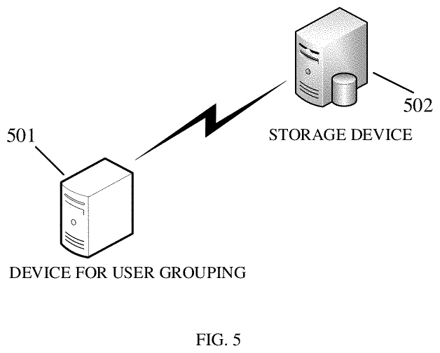

FIG. 4 is a schematic structural diagram illustrating an equipment for user grouping provided in implementations of the disclosure. As illustrated in FIG. 4, the equipment for user grouping includes a processor 401 and a memory 402. Optionally, the equipment for user grouping further includes a communication interface 403. The processor 401, the memory 402, and the communication interface 403 are connected to each other via a bus or in other ways. FIG. 4 illustrates a scenario the processor 401, the memory 402, and the communication interface 403 are connected to each other via a bus. The communication interface 403 can be controlled by the processor to send and receive messages. The memory 402 is configured to store computer programs. The computer programs include program instructions. The processor 401 is configured to execute the program instructions stored in the memory 402. The processor 401 is configured to call the program instructions to carry out the following actions. Net benefits of multiple users in a target project are obtained. According to the net benefits of the multiple users in the target project and a solution of the target project, a net-benefit coefficient corresponding to the solution is determined. For each grouping variable of the target project, a fluctuation value corresponding to the grouping variable is determined according to the net-benefit coefficient. The multiple users are divided into multiple user groups according to a grouping variable with the largest fluctuation value. For each user group obtained by division, a fluctuation value corresponding to each grouping variable of the target project is determined according to a net-benefit coefficient and users in the user group are divided according to a grouping variable with the largest fluctuation value, until a user group meeting a preset condition is obtained.

In some implementations, the processor 401 configured to determine, according to the net benefits of the multiple users in the target project and the solution of the target project, the net-benefit coefficient corresponding to the solution is configured to: fit a net-benefit parameter model in all groups, where the all groups are user groups before the first division, and the net-benefit parameter model is a regression model; and obtain the net-benefit coefficient corresponding to the solution by processing the net benefits in the target project and the solution of the target project with the net-benefit parameter model.

In some implementations, the processor 401 configured to determine, according to the net benefits of the multiple users in the target project and the solution of the target project, the net-benefit coefficient corresponding to the solution is configured to: obtain a net-benefit coefficient table from a block-chain, where the net-benefit coefficient table represents a correspondence among net benefits in a project, a solution of the project, and a net-benefit coefficient; and determine the net-benefit coefficient corresponding to the solution from the net-benefit coefficient table, according to the net benefits in the target project and the solution of the target project.

In some implementations, the processor 401 is further configured to determine, according to a pre-stored correspondence between projects and grouping variables, multiple grouping variables corresponding to the target project. The processor 401 configured to determine, for each grouping variable of the target project, the fluctuation value corresponding to the grouping variable according to the net-benefit coefficient is configured to: for each of the multiple grouping variables of the target project, determine the fluctuation value corresponding to the grouping variable according to the net-benefit coefficient, the net benefits, and a fluctuation function, where the fluctuation value is indicative of a degree of instability of the grouping variable relative to the net benefits.

In some implementations, the processor 401 configured to divide the multiple users into the multiple user groups according to the grouping variable with the largest fluctuation value is configured to: determine a grouping critical value corresponding to the grouping variable with the largest fluctuation value according to a greedy algorithm; and divide the multiple users into the multiple user groups according to the grouping critical value.

In some implementations, the preset condition includes at least one of the following. A significance value corresponding to a fluctuation value is greater than a significance threshold. The number of user groups obtained is greater than a first number threshold. The number of users in at least one of the user groups obtained is less than a second number threshold.

In some implementations, the target project is a target disease, and the solution of the target project is a treatment plan for the target disease. After the user group meeting the preset condition is obtained, the processor 401 is further configured to: determine a user group with the highest net benefit from all user groups obtained, where users in the same user group have the same net benefit; and recommend the treatment plan to users in the user group with the highest net benefit.

It should be understood that, in implementations of the disclosure, the processor 401 may be a central processing unit (CPU). The processor 401 may also be a general-purpose processor, a digital signal processor (DSP), an application specific integrated circuit (ASIC), a field-programmable gate array (FPGA) or other programmable logic devices, a discrete gate or transistor logic device, a discrete hardware component, etc. The general-purpose processor may be a microprocessor, or may also be any conventional processor or the like.

The memory 402 may include a read-only memory (ROM) and a random access memory (RAM). The memory 402 is configured to provide instructions and data to the processor 401. A part of the memory 402 may also include a non-transitory random access memory. For example, the memory 402 may also store net benefits of multiple users in a target project.

The communication interface 403 may include an input device and/or an output device. For example, the input device may be a control panel, a microphone, a receiver, or the like, and the output device may be a display screen, a transmitter, or the like, which is not limited in the disclosure.

In specific implementations, the processor 401, the memory 402, and the communication interface 403 described in implementations of the disclosure can perform the operations of the method implementations described with reference to FIG. 1 or FIG. 2, and can implement the device for user grouping described in implementations of the disclosure, which will not be repeated herein.

According to implementations of the disclosure, a system for user grouping is further provided. FIG. 5 is a schematic structural diagram illustrating a system for user grouping provided in implementations of the disclosure. As illustrated in FIG. 5, the system for user grouping may include a device 501 for user grouping and a storage device 502. The storage device 502 is configured to store data involved in a user grouping process, such as net benefits, information of user groups after grouping, net-benefit coefficients, projects and/or solutions, etc., which is not limited in the disclosure. The device 501 for user grouping can obtain data from the storage device or store data in the storage device. The device for user grouping can be configured to perform all or part of the operations of the foregoing method, or configured to implement functions of the foregoing device or the device for user grouping, which will not be repeated herein.

According to implementations of the disclosure, a computer-readable storage medium is further provided. The computer-readable storage medium stores computer programs. The computer programs include program instructions which, when executed by a processor, are operable to perform all or part of the operations of the method for user grouping in the method implementations, such as performing all or part of the operations which is performed by the device for user grouping (e.g., a server), which will not be repeated herein.

Optionally, the storage medium of the disclosure, such as a computer-readable storage medium, may be a non-transitory storage medium, which is not limited in the disclosure.

According to implementations of the disclosure, a computer program product is further provided. The computer program product includes computer program codes which, when run on a computer, cause the computer to perform the operations of the method for user grouping of the method implementations.

In some implementations, the computer-readable storage medium mainly includes a program storing region and a data storing region. The program storing region may store an operating system, application programs required for at least one function and so on. The data storing region may store data created according to use of a block-chain node, and so on.

The block-chain in the disclosure is a new application mode of computer technology such as distributed data storage, point-to-point transmission, consensus mechanism, and encryption algorithm. Block-chain is essentially a decentralized database. Block-chain is a series of data blocks associated with each other using cryptographic methods. Each data block contains a batch of information of network transactions, to verify the validity of the information (anti-counterfeiting) and generate the next block. Block-chain may include a block-chain underlying platform, a platform product service layer, and an application service layer.

It will be understood by those of ordinary skill in the art that all or part of the operations of the method of the foregoing implementations may be accomplished by means of programs to instruct associated hardware. The programs may be stored in a computer-readable memory. The programs, when executed, are operable to perform the operations of the method of the foregoing implementations. The memory may be a magnetic disk, an optical disc, a ROM, a RAM, or the like.

The foregoing description merely depicts some illustrative implementations of the disclosure, which however are not intended to limit the scope of the claims of the disclosure. Those of ordinary skill in the art can understand that implementing of all or part of the processes of the foregoing implementations, and equivalent changes made in accordance with the claims of the disclosure still fall within the scope of the disclosure.

What is claimed is:

1. A method for user grouping, comprising:
obtaining net benefits of a plurality of users in a target project;
determining, according to the net benefits of the plurality of users in the target project and a solution of the target project, a net-benefit coefficient corresponding to the solution;
for each grouping variable of the target project, determining a fluctuation value corresponding to the grouping variable according to the net-benefit coefficient;
dividing the plurality of users into a plurality of user groups according to a grouping variable with the largest fluctuation value; and
for each user group obtained by division, determining a fluctuation value corresponding to each grouping variable of the target project according to a net-benefit coefficient and dividing users in the user group according to a grouping variable with the largest fluctuation value, until a user group meeting a preset condition is obtained.

2. The method of claim 1, wherein determining, according to the net benefits of the plurality of users in the target project and the solution of the target project, the net-benefit coefficient corresponding to the solution comprises:
fitting a net-benefit parameter model in the plurality of users, wherein the net-benefit parameter model is a regression model; and
obtaining the net-benefit coefficient corresponding to the solution by processing the net benefits in the target project and the solution of the target project with the net-benefit parameter model.

3. The method of claim 1, wherein determining, according to the net benefits of the plurality of users in the target project and the solution of the target project, the net-benefit coefficient corresponding to the solution comprises:
obtaining a net-benefit coefficient table from a block-chain, wherein the net-benefit coefficient table represents a correspondence among net benefits in a project, a solution of the project, and a net-benefit coefficient; and
determining the net-benefit coefficient corresponding to the solution from the net-benefit coefficient table, according to the net benefits in the target project and the solution of the target project.

4. The method of claim 1, further comprising:
before determining, for each grouping variable of the target project, the fluctuation value corresponding to the grouping variable according to the net-benefit coefficient,
determining, according to a pre-stored correspondence between projects and grouping variables, a plurality of grouping variables corresponding to the target project; and
wherein determining, for each grouping variable of the target project, the fluctuation value corresponding to the grouping variable according to the net-benefit coefficient comprises:
for each of the plurality of grouping variables of the target project, determining the fluctuation value corresponding to the grouping variable according to the net-benefit coefficient, the net benefits, and a fluctuation function, wherein the fluctuation value is indicative of a degree of instability of the grouping variable relative to the net benefits.

5. The method of claim 1, wherein dividing the plurality of users into the plurality of user groups according to the grouping variable with the largest fluctuation value comprises:
determining a grouping critical value corresponding to the grouping variable with the largest fluctuation value according to a greedy algorithm; and
dividing the plurality of users into the plurality of user groups according to the grouping critical value.

6. The method of claim 1, wherein the preset condition comprises at least one of:
a significance value corresponding to a fluctuation value being greater than a significance threshold;
the number of user groups obtained being greater than a first number threshold; or
the number of users in at least one of the user groups obtained being less than a second number threshold.

7. The method of claim 1, wherein the target project is a target disease and the solution of the target project is a treatment plan for the target disease, and wherein the method further comprises:
after the user group meeting the preset condition is obtained,
determining a user group with the highest net benefit from all user groups obtained, wherein users in the same user group have the same net benefit; and
recommending the treatment plan to users in the user group with the highest net benefit.

8. An equipment for user grouping, comprising:
a processor; and
a memory, coupled with the processor and configured to store computer programs;
the computer programs comprising program instructions which are called by the processor and cause the processor to:
obtain net benefits of a plurality of users in a target project;
determine, according to the net benefits of the plurality of users in the target project and a solution of the target project, a net-benefit coefficient corresponding to the solution;
for each grouping variable of the target project, determine a fluctuation value corresponding to the grouping variable according to the net-benefit coefficient;
divide the plurality of users into a plurality of user groups according to a grouping variable with the largest fluctuation value; and
for each user group obtained by division, determine a fluctuation value corresponding to each grouping variable of the target project according to a net-benefit coefficient and divide users in the user group according to a grouping variable with the largest fluctuation value, until a user group meeting a preset condition is obtained.

9. The equipment of claim 8, wherein the processor configured to determine, according to the net benefits of the plurality of users in the target project and the solution of the target project, the net-benefit coefficient corresponding to the solution is configured to:
fit a net-benefit parameter model in the plurality of users, wherein the net-benefit parameter model is a regression model; and
obtain the net-benefit coefficient corresponding to the solution by processing the net benefits in the target project and the solution of the target project with the net-benefit parameter model.

10. The equipment of claim 8, wherein the processor configured to determine, according to the net benefits of the plurality of users in the target project and the solution of the target project, the net-benefit coefficient corresponding to the solution is configured to:
obtain a net-benefit coefficient table from a block-chain, wherein the net-benefit coefficient table represents a correspondence among net benefits in a project, a solution of the project, and a net-benefit coefficient; and
determine the net-benefit coefficient corresponding to the solution from the net-benefit coefficient table, according to the net benefits in the target project and the solution of the target project.

11. The equipment of claim 8, wherein:
the processor is further configured to determine, according to a pre-stored correspondence between projects and grouping variables, a plurality of grouping variables corresponding to the target project, before determining, for each grouping variable of the target project, the fluctuation value corresponding to the grouping variable according to the net-benefit coefficient;
the processor configured to determine, for each grouping variable of the target project, the fluctuation value corresponding to the grouping variable according to the net-benefit coefficient is configured to:
for each of the plurality of grouping variables of the target project, determine the fluctuation value corresponding to the grouping variable according to the net-benefit coefficient, the net benefits, and a fluctuation function, wherein the fluctuation value is indicative of a degree of instability of the grouping variable relative to the net benefits.

12. The equipment of claim 8, wherein the processor configured to divide the plurality of users into the plurality of user groups according to the grouping variable with the largest fluctuation value is configured to:
determine a grouping critical value corresponding to the grouping variable with the largest fluctuation value according to a greedy algorithm; and
divide the plurality of users into the plurality of user groups according to the grouping critical value.

13. The equipment of claim 8, wherein the preset condition comprises at least one of:
a significance value corresponding to a fluctuation value being greater than a significance threshold;
the number of user groups obtained being greater than a first number threshold; or
the number of users in at least one of the user groups obtained being less than a second number threshold.

14. The equipment of claim 8, wherein the target project is a target disease and the solution of the target project is a treatment plan for the target disease, and wherein the processor is further configured to:
after the user group meeting the preset condition is obtained,
determine a user group with the highest net benefit from all user groups obtained, wherein users in the same user group have the same net benefit; and
recommend the treatment plan to users in the user group with the highest net benefit.

15. A non-transitory computer-readable storage medium, storing computer programs comprising program instructions which, when executed by a processor, cause the processor to carry out the following actions:
obtaining net benefits of a plurality of users in a target project;
determining, according to the net benefits of the plurality of users in the target project and a solution of the target project, a net-benefit coefficient corresponding to the solution;
for each grouping variable of the target project, determining a fluctuation value corresponding to the grouping variable according to the net-benefit coefficient;
dividing the plurality of users into a plurality of user groups according to a grouping variable with the largest fluctuation value; and
for each user group obtained by division, determining a fluctuation value corresponding to each grouping variable of the target project according to a net-benefit coefficient and dividing users in the user group according to a grouping variable with the largest fluctuation value, until a user group meeting a preset condition is obtained.

16. The non-transitory computer-readable storage medium of claim 15, wherein the program instructions that cause the processor to carry out the actions of determining, according to the net benefits of the plurality of users in the target project and the solution of the target project, the net-benefit coefficient corresponding to the solution cause the processor to carry out the following actions:
fitting a net-benefit parameter model in the plurality of users, wherein the net-benefit parameter model is a regression model; and
obtaining the net-benefit coefficient corresponding to the solution by processing the net benefits in the target project and the solution of the target project with the net-benefit parameter model.

17. The non-transitory computer-readable storage medium of claim 15, wherein the program instructions that cause the processor to carry out the actions of determining, according to the net benefits of the plurality of users in the target project and the solution of the target project, the net-benefit coefficient corresponding to the solution cause the processor to carry out the following actions:
obtaining a net-benefit coefficient table from a block-chain, wherein the net-benefit coefficient table represents a correspondence among net benefits in a project, a solution of the project, and a net-benefit coefficient; and
determining the net-benefit coefficient corresponding to the solution from the net-benefit coefficient table, according to the net benefits in the target project and the solution of the target project.

18. The non-transitory computer-readable storage medium of claim 15, wherein the program instructions further cause the processor to carry out the following actions:
before determining, for each grouping variable of the target project, the fluctuation value corresponding to the grouping variable according to the net-benefit coefficient,
determining, according to a pre-stored correspondence between projects and grouping variables, a plurality of grouping variables corresponding to the target project; and
wherein the program instructions that cause the processor to carry out the actions of determining, for each grouping variable of the target project, the fluctuation value corresponding to the grouping variable according to the net-benefit coefficient cause the processor to carry out the following actions:
for each of the plurality of grouping variables of the target project, determining the fluctuation value corresponding to the grouping variable according to the net-benefit coefficient, the net benefits, and a fluctuation function, wherein the fluctuation value is indicative of a degree of instability of the grouping variable relative to the net benefits.

19. The non-transitory computer-readable storage medium of claim 15, wherein the preset condition comprises at least one of:
a significance value corresponding to a fluctuation value being greater than a significance threshold;
the number of user groups obtained being greater than a first number threshold; or
the number of users in at least one of the user groups obtained being less than a second number threshold.

20. The non-transitory computer-readable storage medium of claim 15, wherein the target project is a target disease and the solution of the target project is a treatment plan for the target disease, and wherein the program instructions further cause the processor to carry out the following actions:
after the user group meeting the preset condition is obtained,
determining a user group with the highest net benefit from all user groups obtained, wherein users in the same user group have the same net benefit; and
recommending the treatment plan to users in the user group with the highest net benefit.
